(19) United States
(12) Patent Application Publication
(10) Pub. No.: US 2023/0191517 A1
(43) Pub. Date: Jun. 22, 2023

(54) METHOD FOR ASSEMBLING A ZIRCONIA PART TO A TITANIUM ELEMENT

(71) Applicants: COMMISSARIAT À L'ÉNERGIE ATOMIQUE ET AUX ÉNERGIES ALTERNATIVES, Paris (FR); OTICON MEDICAL A/S, SMORUM (DK); INSTITUT POLYTECHNIQUE DE GRENOBLE, GRENOBLE CEDEX 1 (FR); UNIVERSITÉ GRENOBLE ALPES, SAINT-MARTIN-D'HERES (FR); CENTRE NATIONAL DE LA RECHERCHE SCIENTIFIQUE, PARIS CEDEX 16 (FR)

(72) Inventors: Marie Fischer, Grenoble (FR); Valerie Chaumat, Grenoble (FR); Camille Godinot, Grenoble (FR); Fiqiri Hodaj, GRENOBLE (FR); Ania Merlet, FAYENCE (FR); Guillaume Tourrel, SAINT-JEANNET (FR)

(21) Appl. No.: 18/082,711

(22) Filed: Dec. 16, 2022

(30) Foreign Application Priority Data
Dec. 17, 2021 (FR) 2113878

(51) Int. Cl. B23K 1/00; A61N 1/05; B23K 1/20; B23K 35/30; C22C 5/02

(52) U.S. Cl. CPC B23K 1/0008; A61N 1/0541; B23K 1/20; B23K 35/3013; C22C 5/02; B23K 2103/52

(57) ABSTRACT

A method for assembling a zirconia part to a titanium element with braze, the method comprising the following steps: coating a surface of the titanium element with a niobium layer, positioning a braze between the zirconia part and the niobium, the braze being of gold or a gold alloy, heating the whole to a temperature higher than the melting temperature of the braze, and then cooling the whole, whereby an assembly comprising the zirconia part and the titanium element assembled by a brazing joint comprising a first portion of gold or a gold alloy, a second portion formed by a reaction layer comprising intermetallics of the AuNbTi system, and a third portion formed by an oxide reaction layer is obtained.

### CROSS-REFERENCE TO RELATED APPLICATIONS

This application claims priority from French Patent Application No. 2113878 filed on Dec. 17, 2021. The content of this application is incorporated herein by reference in its entirety.

### TECHNICAL FIELD

The present invention generally relates to the field of methods for assembling ceramic parts with metal elements by brazing.

The invention relates to a method for assembling a zirconia part to a titanium element by brazing.

The invention also relates to an assembly obtained by such a method.

The invention finds applications in many industrial fields, and in particular in the biomedical field, for example for the manufacture of hearing implants, in particular cochlear implants, or pacemakers, or orthopaedic or dental implants, or in the field of watchmaking.

The invention is particularly interesting because it allows strong interfaces to be created when assembling and thus allows parts with very good mechanical properties to be obtained.

### STATE OF PRIOR ART

Brazing is an assembling technique that consists in positioning a filler material, called braze or brazing alloy, between or in proximity to the parts to be assembled, and then melting it to fill the gap between the parts. After cooling, it forms a bond between the parts to be brazed. The parts are thus assembled together by a brazing joint. The filler material has a lower melting temperature than the materials to be brazed, so that the parts to be assembled do not melt during the brazing method. In order to achieve proper brazing, it is necessary to have good wetting of the surfaces to be brazed by the liquid braze and formation of strong bonds at the braze/parts to be brazed interfaces.

Since the 2000s, zirconia/metal brazing and in particular zirconia/titanium brazing has been widely studied in the literature for various applications.

However, it is known that zirconia is not wettable by the liquid metals Au, Ag, Cu, Ni.

In order to overcome this drawback, one solution is to use a braze formed by a matrix consisting of one of these metals and an active element such as Ti or Zr. The active element allows wetting of the zirconia. Widely used brazes are Ag—Cu—Ti alloys.

Another solution is to first deposit the active element onto the zirconia (PVD, sputtering, etc.) and then perform brazing with a braze that does not contain an active element (for example Ag—Cu). However, these Ag—Cu type brazes are not biocompatible, nor are active brazes consisting of this Ag—Cu matrix and an active element (Ag—Cu—Ti brazes).

Otherwise, when the metal element to be assembled to the zirconia is titanium, it is possible to perform zirconia/titanium brazing using a braze containing no active element and without prior metallisation. Indeed, in this case, the active element Ti is provided by the titanium substrate by dissolution and diffusion of Ti through the braze. This is enriched with the titanium element which will react with zirconia to form a wettable reaction product (titanium oxide). Therefore, this allows wetting of the ceramic and also tack of the braze to zirconia.

In order to have a biocompatible braze, it is possible to choose a gold braze [1-5] for brazing zirconia with titanium. Brazes described can be performed without metallisation of zirconia [1-4] or with metallisation of zirconia [5].

For example, in references [1-2], brazing of zirconia and titanium is performed with a 25 μm thick gold foil under vacuum at 1065-1100° C. The formation of a titanium oxide TiₓO_{y} at the interface with zirconia and intermetallics of the Ti—Au system on the Ti side are observed.

The effect of brazing temperature and brazing plateau duration on the interfaces and microstructure in ZrO₂/Au/Ti joints, obtained with 50 μm thick gold foils under vacuum (1.3×10⁻³ Pa) at 1110-1190° C. for 5-30 min was also studied [3]. The microstructure detected on the Ti side consists of 4 intermetallic compounds of the Ti—Au system, whose overall thickness varies with holding time and temperature (about 34 μm for 10 min at 1150° C.). On the zirconia side, a thin reaction layer of TiₓO_{y} is detected and its thickness varies with time and temperature. An optimum shear stress of 35 MPa was obtained on a brazed joint at 1150° C. for 10 min.

Finally, Fischer et al [4] studied wetting of zirconia by Au—Ti alloys and ZrO₂/Au/Ti brazing. Liquid gold does not wet zirconia. The addition of a few percent of Ti in gold allows wetting of zirconia, due to the formation of a reaction layer TiₓO_{y} at the Au/zirconia interface. The contact angle (θ) of Au—Ti alloys on zirconia decreases as the Ti content in gold increases, until reaching θ=44±3° at 1150° C. with 3% by mass of Ti in Au. Brazing of titanium-containing zirconia with pure gold was successfully achieved at 1100° C. for 15 min, using the active element Ti, from the Ti substrate. In the ZrO₂/Au/Ti assembly configuration, the active element (Ti) in liquid gold is provided first by dissolution of the titanium substrate and then by diffusion of Ti through the different layers of Au—Ti intermetallic compounds formed at the Ti/Au interface. In parallel, titanium diffuses through the liquid gold to the ZrO₂/Au interface where it reacts with zirconia and forms a wettable TiₓO_{y} reaction product, resulting in proper wetting of the zirconia by liquid gold.

However, the mechanical strength of the joints formed by ZrO₂/Au/Ti direct brazing remains insufficient.

In document [5], the zirconia/metal brazing method is carried out with gold braze. Zirconia is previously metallised with a first titanium metallisation layer, followed by a second niobium metallisation layer. The titanium layer ensures tack to zirconia, and also allows tack of the second niobium layer. The latter would allow tack of the gold braze which adheres to the metal element (for example titanium).

However, in this method, in order to promote tack of the first titanium metallisation layer to zirconia, the surface condition of the ceramic has to be impaired to increase its surface roughness, for example by a sandblasting technique. In addition, this method requires a stress relief heat treatment carried out under load on the metal substrate before brazing.

These different steps (impairing the zirconia surface and stress relief heat treatment) and the need for double metallisation make the method more complex.

### DISCLOSURE OF THE INVENTION

One purpose of the present invention is to provide a method for assembling a zirconia part overcoming the drawbacks of prior art and making it possible to obtain an assembly with good mechanical strength.

For this, the present invention provides a method for assembling a zirconia part to a titanium part with braze, the method comprising the following steps:

preferably, cleaning the zirconia part and/or the titanium element and/or the braze, for example this may be a step during which titanium is chemically etched beforehand,

coating a surface to be brazed of the titanium element with a niobium layer,

positioning braze between the zirconia part and the niobium layer, the braze being of gold or a gold alloy,

heating the whole to a temperature higher than the melting temperature of the braze, and then cooling the whole, whereby an assembly comprising the zirconia part and the titanium element assembled by a brazing joint comprising a first portion of gold or a gold alloy, a second portion formed by a reaction layer comprising intermetallics of the AuNbTi system, and a third portion formed by a reaction layer of oxide is obtained.

The reaction layer comprising intermetallics of the AuNbTi system is positioned between the first part made of gold or gold alloy and the titanium element. It results from the reaction of the niobium layer with gold in the braze and with titanium in the titanium element. It comprises or consists of intermetallics of the AuNbTi ternary system. For example, it is formed by intermetallics of the AuNbTi ternary system in the case of a pure gold braze or it is formed mainly of intermetallics of the AuNbTi ternary system in the case of a gold alloy braze.

The first portion may have the same composition as the braze or be slightly titanium enriched with respect to the initial composition of the braze (typically a composition with 0.1 to 5% by mass of titanium, preferably 0.1 to 2% by mass of titanium).

In addition, at the end of the method, an oxide reaction layer is present between the first part made of gold or gold alloy of the brazing joint and the zirconia part. This is a thin oxide reaction layer. The thickness of this oxide layer is small (usually less than 2 μm or even submicron-sized, i.e. less than 1 μm). Preferably, the oxide layer is between 100 nm and 300 nm thick.

The presence of this layer allows wetting and tack between gold and zirconia during brazing.

By way of illustrating and not limiting purposes, the oxide layer may be:

a layer of titanium oxide TiₓO_{y},

a layer comprising a mixture of several oxides, in particular a mixture of titanium oxides and metal(s) present in the composition of the gold alloy (for example a mixture of titanium and zirconium oxides in the case of AuxZr braze), or

a layer of a mixed oxide of titanium and metal(s) present in the composition of the gold alloy (for example a mixed oxide of titanium and zirconium in the case of AuxZr braze).

By zirconia part, it is meant here and hereafter a zirconium oxide (ZrO₂) part or a zirconium oxide based part. Preferably, the zirconium oxide-based part is an yttria-stabilised zirconia part. The yttria-stabilised zirconia can for example be a Y-TZP (‘Yttrium-Tetragonal Zirconia Polycrystal’) zirconia stabilised with 3 mole % of Y₂O₃. Finally, the zirconia-based part can be a part made of an alumina-zirconia composite material, such as ZTA (“Zirconia Toughened Alumina”) with an alumina matrix and ATZ (“Alumina Toughened Zirconia”) with a zirconia matrix. These composites combine properties of zirconia and alumina.

The invention fundamentally differs from prior art, on the one hand, by the deposition of a niobium layer onto the titanium part to metalline it, and on the other hand, by the absence of metallisation of the zirconia part.

This metallisation layer on the titanium element plays a modifying role in the formation of the Ti/Au interface. Indeed, during the brazing method, the presence of niobium modifies the Ti/Au interface, that is the morphology, thickness and nature of the intermetallics formed at this interface. The fracture surfaces, after mechanical tests, are additionally modified with respect to those obtained on brazed assemblies without titanium metallisation.

The assembly obtained by brazing with the deposition of the Nb layer onto titanium has a very good mechanical strength.

In particular, joints obtained with such a method show, in tensile tests, rupture stress values:

significantly higher than those obtained by direct zirconia/Au/titanium brazing without prior metallisation of titanium, and

very significantly higher than those of brazing a Ti+Nb bilayer metallised zirconia with a titanium element.

According to one advantageous alternative, the braze is of gold.

According to another advantageous alternative, the gold braze is a gold alloy comprising between 96 and 99.5% by mass of gold.

Advantageously, the braze is a gold alloy, the gold alloy comprising gold, titanium or zirconium and optionally nickel. For example, the gold alloy comprises gold, titanium and nickel.

According to this advantageous alternative, the braze comprises between 0.5 and 4% by mass of titanium or zirconium and possibly nickel.

Preferably, the braze comprises 0.5% to 3% by mass of titanium.

Advantageously, the braze is a braze of the composition Au3Ni0.6Ti (% by mass), AuxTi or AuxZr with x between 0.5 and 3% by mass. For example, it can be 0.5% Ti-99.5% Au.

Advantageously, the braze is a braze foil with a thickness of between 25 μm and 200 μm, preferably between 50 and 100 μm.

Advantageously, step d) is carried out under a secondary vacuum or under a protective atmosphere (under reducing atmosphere or under neutral gas).

The method is simple to implement as only one metallisation step is required and there is no need to increase the surface roughness of zirconia. The method can be implemented with different brazes: pure gold or gold alloy brazes.

The invention also relates to an assembly obtained by the previously described method successively comprising:

a zirconia part,

a brazing joint (or brazed joint) comprising a first portion of gold or a gold alloy, a second portion formed by a reaction layer comprising or consisting of intermetallics of the AuNbTi system, and a third portion formed by an oxide reaction layer,

a titanium element.

In other words, the brazing joint comprises, and preferably consists of, from the zirconia part to the titanium element: the oxide reaction layer, the gold or gold alloy portion and the intermetallic reaction layer of the AuNbTi system.

Preferably, the brazing joint comprises a first portion of gold or gold alloy AuNiTi, AuTi or AuTiZr and a second portion formed by a reaction layer comprising or consisting of intermetallics of the AuNbTi system on the titanium side. The brazing joint further comprises a third portion formed by a reaction layer of oxide (titanium oxide TiₓO_{y} or mixture of oxides including titanium oxide, or mixed titanium oxide) on the zirconia side.

According to one advantageous embodiment, the brazing joint is obtained from a gold braze. The brazing joint comprises gold on the one hand and a reaction layer of intermetallics of the AuNbTi system on the titanium side on the other hand. The gold portion of the brazing joint can be slightly enriched with titanium. The titanium comes from the titanium substrate. The brazing joint further comprises a layer of titanium oxide TiₓO_{y} on the zirconia side.

According to another advantageous alternative embodiment, the brazing joint is obtained from a braze of a gold alloy having composition Au3Ni0.6Ti (% by mass). The brazing joint comprises on the one hand a gold alloy (AuNiTi) and on the other hand a reaction layer mainly formed by intermetallics of the ternary system AuNbTi on the titanium side. The brazing joint further comprises a layer of titanium oxide TiₓO_{y} on the zirconia side.

According to another advantageous alternative embodiment, the brazing joint is obtained from a braze of gold alloy having composition AuxTi or AuxZr (with x between 0.5 and 3% by mass). The brazing joint comprises a gold alloy (AuTi or AuTiZr) on the one hand and a reaction layer mainly formed by intermetallics of the AuNbTi ternary system on the titanium side on the other hand. The brazing joint further comprises a reaction layer of oxide (titanium oxide TiₓO_{y} in the case of AuxTi; titanium oxide TiₓO_{y} or a mixture of oxides including titanium oxide, or mixed titanium oxide in the case of AuxZr) on the zirconia side.

In these different alternative embodiments, the oxide layer is a thin oxide layer. Preferably, it has a thickness between 100 nm and 300 nm.

Advantageously, zirconia is an yttria-stabilised zirconia or an alumina-zirconia composite.

Assemblies obtained with this method have a very good mechanical strength, in particular from 115 MPa to 250 MPa in tensile strength.

The present invention thus makes it possible to form a zirconia/titanium assembly with good joint filling by the braze, very strong interfaces and good mechanical strength. This assembly is biocompatible in the case of Au, AuxTi and AuxZr brazes.

The invention also relates to a cochlear implant comprising an assembly as hereinbefore defined, the brazing joint of the assembly comprising a first portion of gold or a gold alloy AuTi or AuTiZr, a second portion formed by a reaction layer comprising or consisting of intermetallics of the AuNbTi system, and a third portion formed by an oxide reaction layer.

Further characteristics and advantages of the invention will be apparent from the following further description.

Needless to say that this further description is only given as an illustration of the subject matter of the invention and should in no way be construed as limiting this subject matter.

### BRIEF DESCRIPTION OF THE DRAWINGS

The present invention will be better understood upon reading the description of exemplary embodiments given by way of illustrating and not in any way limiting purposes, with reference to the appended drawings in which:

FIGS. 1A, 1B and 1C schematically represent in cross-section views different steps of a method for manufacturing an assembly of a titanium element with a zirconia part, according to one particular embodiment of the invention.

FIG. 2 is a scanning electron microscope (SEM) picture of an assembly comprising a titanium member initially covered with a niobium layer, a brazing joint (formed from a gold braze) and a zirconia part, according to one particular embodiment of the invention; the presence of two zones in the joint formed is noticed: gold and the reaction layer of the AuNbTi ternary system.

FIG. 3 is an SEM picture of an assembly comprising a titanium element, a brazing joint (formed from a gold braze) and a zirconia part, according to a method of prior art; the presence of two zones in the joint formed is noticed: gold and the reaction layer with 4 intermetallics of the Au—Ti system.

FIG. 4 schematically represents in three dimensions a tensile test piece before brazing comprising two zirconia parts, a titanium element covered on either side with a niobium layer, and two brazes (gold rings), according to one particular embodiment of the invention, the insertion represents a zoom of the centre of the test piece.

FIG. 5 is a photograph of a test piece in a tooling according to one particular embodiment of the invention.

FIGS. 6A and 6B are SEM pictures of a zirconia/Au/titanium braze with double metallisation of the zirconia with Ti+Nb, before and after heat treatment respectively, according to a comparative example.

FIG. 7 is an SEM picture of a Ti+Nb metallised zirconia/gold braze interface, after one brazing cycle, according to a comparative example.

Different parts represented in the figures are not necessarily represented to scale, to make the figures more legible.

The different possibilities (alternatives and embodiments) should be understood as being not exclusive of each other and can be combined with each other.

In addition, in the following description, terms that depend on the orientation, such as “on”, of a structure are applied assuming that the structure is oriented the illustrated way in the figures.

### DETAILED DESCRIPTION OF PARTICULAR EMBODIMENTS

Although this is by no means limiting, the invention particularly finds applications in the biomedical field for the manufacture of hearing implants, and in particular for the manufacture of cochlear implants, for the manufacture of pacemakers, orthopaedic or dental implants, or in the field of watchmaking.

The method for assembling a titanium element 10 to a zirconia part 20 with a brazing material comprises the following steps:

preferably cleaning the titanium element 10 and/or the zirconia part 20, as well as the braze 40,

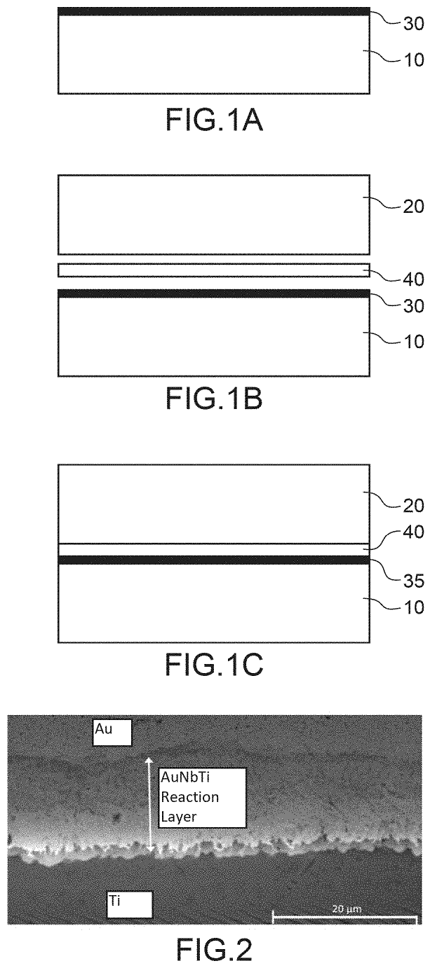

b) covering a surface of the titanium element 10 with a niobium layer 30 (FIG. 1A), titanium possibly being chemically etched beforehand,

c) positioning braze 40 between the zirconia part 20 and the niobium layer 30, the braze 40 being of gold or a gold alloy (FIG. 1B),

d) heating the whole to a temperature higher than the melting temperature of the braze 40 and then cooling the whole, whereby an assembly comprising the zirconia part 20 and the titanium element 10 assembled by a brazing joint comprising a first portion of gold or a gold alloy, a second portion formed by a reaction layer 35 comprising, or consisting of, intermetallics of the AuNbTi ternary system on the titanium side (FIG. 1C) is obtained. An oxide reaction layer (third portion) is also present in the joint on the zirconia side. It is detectable by transmission electron microscopy and, depending on the thickness and/or nature of the oxides, it can also be detected by scanning electron microscopy (SEM).

The zirconia part 20 is of zirconium oxide (ZrO₂), or yttria-stabilised zirconia (ZrO₂—Y₂O₃) or alumina-zirconia composite, such as ZTA (Zirconia Toughened Alumina) with an alumina matrix and ATZ (Alumina Toughened Zirconia) with a zirconia matrix.

The yttria-stabilised zirconia preferably contains between 1 and 6 mole % of Y₂O₃. The yttria-stabilised zirconia is, for example, a Y-TZP (“Yttrium-Tetragonal Zirconia Polycrystal”) zirconia stabilised with 3 mole % of Y₂O₃.

By “between X and Y”, it is understood that bounds X and Y are included.

In the following, the term zirconia will be used, but it is obvious that the term zirconia can be replaced with yttria-stabilised zirconia or alumina-zirconia composite.

For example, for step a), it is preferable to clean the zirconia part 20 to be brazed, the titanium element 10 and the braze 40 in a bath of acetone, and then ethanol with ultrasound.

The surface of the titanium element 10, which is to be metallised, may be subjected to a chemical etching step and/or an ion etching step.

In addition, the zirconia may be heat treated at 1350° C., preferably under air (the so-called roasting method), prior to step c).

During step b), the titanium element 10 to be brazed is first coated with a niobium deposit. This metallisation layer 30 makes it possible to reinforce the Ti/Au interface by modifying, during the brazing method, the morphology, thickness and nature of the intermetallics in the Ti—Au system.

Deposition of the niobium layer 30 is, for example, performed by physical vapour deposition (PVD) or by sputtering. The thickness of the niobium deposited ranges, for example, from a few microns to 20 μm, typically 5 to 10 μm.

The zirconia part 20 is not metallised, that is the braze 40 is in direct contact with the surface of the zirconia part 20 to be assembled.

It is obvious that it is possible to assemble one or more zirconia parts with one or more titanium elements. For example, it is possible to assemble two zirconia parts with one titanium part. In this case, two brazes are implemented and the two faces of the titanium part to be in contact with both brazes are metallised.

Braze 40 is a gold-rich braze such as pure gold or a gold alloy.

By pure gold, it is understood gold with a purity of more than 99% by mass, preferably more than 99.5% by mass.

The gold alloy is, for example, a braze with the composition Au3Ni0.6Ti (% by mass), for example marketed under the reference GoIdABA, or an AuxTi or AuxZr braze with x between 0.5 and 3% by mass.

The melting temperature of the braze is lower than the melting temperature of the titanium element.

The braze 40 is advantageously in the form of a foil.

The thickness of the braze foil is, for example, between 25 and 200 μm, preferably between 50 and 150 μm.

The braze 40 can also be in the form of a powder.

During step c) and during step d), tooling for holding the zirconia part 20, the titanium element 10 and the braze 30 may be used.

During the brazing method (step d), the zirconia part 20 is assembled to the titanium part 10 by melting the braze 40. The whole is heated so that the braze melts. The braze plateau temperature depends on the braze composition. The braze plateau temperature is higher than the melting temperature of the braze 40. Typically, the braze plateau temperature is higher than 1064° C. in the case of pure gold and preferably lower than 1120° C. In the case of commercial braze GoIdABA, the liquidus and solidus are 1030° C. and 1003° C. respectively. The brazing temperature is preferably higher than 1030° C. and lower than 1120° C.

The brazing plateau duration is for example between 0 and 15 minutes, preferably between 30 seconds and 10 minutes.

After the brazing plateau, the whole is cooled to room temperature. The braze solidifies as it cools and the parts are then bonded by the braze solidified.

Metallisation of titanium element 10 by niobium does not lead to the formation of porosities at the Nb metallised titanium/Au brazing interface during heating associated with the brazing cycle, contrary to the case of the metallisation of zirconia (Ti+Nb metallised zirconia/Au interface weakened by the formation of porosities upon heating).

This brazing method can be carried out under a secondary vacuum or under a protective atmosphere (neutral gas or reducing atmosphere). It is preferably carried out in a secondary vacuum furnace.

The assembly obtained by the method described above successively comprises:

a zirconia part 20,

a brazing joint comprising a first portion 40 made of gold or a gold alloy (on the side of the zirconia part 20), a second portion formed by a reaction layer 35 comprising or consisting of intermetallics of the AuNbTi ternary system on the titanium side (layer formed by reaction of the metallisation layer 30 with the braze 40 and with the titanium element 10), and finally a third portion (not represented) formed by an oxide layer on the zirconia side, and

a titanium element 10.

The first portion has the same composition as the braze or a composition corresponding to the composition of the braze slightly enriched with titanium. The titanium content is between 0.1% and 5% by mass of titanium, preferably between 0.1% and 2% Ti.

The reaction layer 35 of intermetallics is positioned between the first gold or gold alloy portion 40 and the titanium element. The reaction layer 35 of intermetallics has, for example, a thickness of 5 μm to 30 μm.

The thin oxide layer is positioned between the first gold or gold alloy part 40 and the zirconia part. The thin oxide layer has, for example, a thickness between 100 nm and 2 μm.

Advantageously, the brazing joint comprises a gold alloy AuNiTi, AuTi or AuTiZr, a reaction layer 35 comprising or consisting of intermetallics of the AuNbTi ternary system, and possibly a thin oxide layer on the zirconia side.

According to one advantageous alternative embodiment, the zirconia is yttria-stabilised zirconia.

According to another advantageous alternative embodiment, the zirconia is an alumina-zirconia composite.

The invention is particularly interesting for forming hearing implants.

In particular, a cochlear implant comprising an assembly as defined above could be manufactured, the brazing joint of the assembly comprising, on the one hand, gold or a gold alloy AuTi or AuTiZr and, on the other hand, a reaction layer 35 comprising or consisting of intermetallics of the AuNbTi ternary system. A thin oxide reaction layer is also present on the zirconia side.

### Illustrative and Non-Limiting Examples of One Embodiment

Several non-limiting exemplary embodiments as well as comparative tests will now be described.

In the following examples and tests, zirconia is 3 mole % yttria-stabilised zirconia.

### EXAMPLE 1

### Making Assemblies by Zirconia/Au/Titanium Brazing with Nb Deposits having Thickness X μm (X=5, 10, 15 μm) on Titanium

Three assemblies «zirconia disc 20 (ϕ15 mm, h 2 mm)/titanium disc 10 (ϕ15 mm, h 2 mm)» have been made by brazing with a pure gold foil 40. The titanium discs are previously metallised with a niobium layer 30. Three different thicknesses have been tested: 5 μm, 10 μm and 15 μm.

The gold foil 40 is cut to form a disc with a diameter of 15 mm and a thickness of 100 μm.

The following steps have been performed to make these assemblies:

Step 1: Cleaning the zirconia, titanium and gold parts to be brazed in a bath of acetone, and then ethanol with ultrasound, wherein titanium has been chemically etched beforehand.

Step 2: Metallising the surfaces to be brazed of the three titanium parts by performing ion etching of the titanium followed by a niobium deposition by PVD. The thickness of niobium is respectively 5 μm for assembly n°1, 10 μm for assembly n°2 and 15 μm for assembly n°3.

Step 3: Depositing the pure gold brazing foil between the zirconia and titanium parts to be assembled. The niobium-coated side of titanium is that positioned towards the braze.

Step 4: Placing the three assemblies formed by the parts and brazes in a secondary vacuum brazing furnace. A small 23 g W mass has been placed on each assembly to hold the assembly in the furnace.

Step 5: Heating the mixture to 1080° C. for a few minutes.

Step 6: After the brazing plateau, the assembly is cooled to room temperature and the parts are then bonded with the braze solidified.

The parts have been cut, coated with epoxy resin and polished. The joints are properly filled with braze, there is no lack of braze, that is there are no holes in the braze. The interfaces are not cracked.

It can be noted that the titanium/gold interface (FIG. 2) is not made up of successive layers of Ti—Au intermetallics, unlike the case of direct brazing without titanium metallisation by niobium.

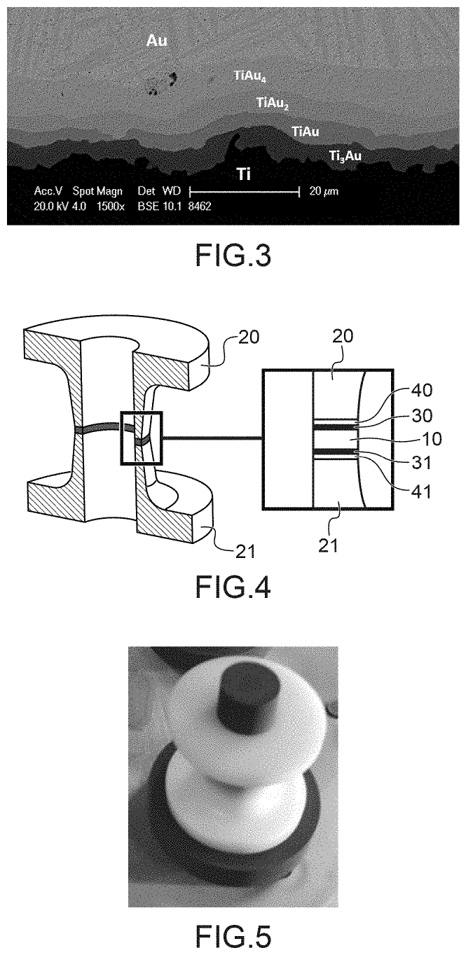

Indeed, by way of comparison, an assembly with a direct brazing of zirconia/Au/titanium without titanium metallisation by niobium has been manufactured and then characterised. The presence of 4 parallel and successive layers of intermetallic Ti₃Au, TiAu, TiAu₂, TiAu₄ (FIG. 3) is observed.

### EXAMPLE 2

### Manufacture of Tensile Test Pieces by Zirconia/Au/Titanium Brazing with a 5 μm Thick Nb Deposit, and Mechanical Tests

Tensile test pieces have been brazed based on the ASME F19-64 standard used for characterising ceramic/metal assemblies. A diagram sets forth this type of test piece in the case of zirconia/Au/titanium brazing (FIG. 4). In this diagram, the titanium ring 10 can be seen in the centre of the part. On either side of the ring, gold brazing rings 40, 41, and finally the zirconia parts 20, 21 are placed. Each side of the titanium ring 10 is covered with a niobium layer 30, 31. The whole is held in a tooling during the brazing operation so that there is no misalignment between the parts (FIG. 5).

The brazing has been carried out as follows:

Step 1: Cleaning the zirconia, titanium and gold parts to be brazed in a bath of acetone, and then ethanol with ultrasound, wherein titanium has been chemically etched beforehand.

Step 2: Metallising the 2 surfaces to be brazed of the titanium rings by performing ion etching of titanium followed by a niobium deposition by PVD. The niobium thickness is 5 μm.

Step 3: Depositing 2 rings of pure gold braze between the zirconia and titanium parts to be assembled (zirconia/Au/titanium/Au/zirconia).

Step 4: Placing each sample to be brazed in a hold tooling and install the whole in a secondary vacuum brazing furnace.

Step 5: Heating the mixture to 1080° C. for a few minutes.

Step 6: After the brazing plateau, the whole is cooled to room temperature and the test pieces are then bonded by the braze solidified.

Four test pieces have thus been produced. They have been subjected to tensile tests. The rupture stress values are measured. An average rupture stress of 179 MPa has been obtained.

### COMPARATIVE EXAMPLE 3

### Making Zirconia/Titanium Assemblies and Tensile Test Pieces by Zirconia/Au/Titanium Brazing without any Metallisation of the Substrates to be Brazed, and Mechanical Tests

Tensile test pieces have been brazed according to the same protocol as for example 2, but without making titanium metallisation nor zirconia metallisation. These test pieces have been subjected to the tensile test and the rupture stress values have been measured. After brazing at 1080° C. and a few minutes of a plateau, an average rupture stress of 97 MPa is obtained, which is significantly lower than the stress obtained with titanium metallisation by niobium.

Rupture occurs between the intermetallics TiAu₂ and TiAu₄.

### COMPARATIVE EXAMPLE 4

### Making Zirconia/Titanium Assemblies and Tensile Test Pieces by Zirconia/Au/Titanium Brazing with Double Metallisation of Zirconia by Ti+Nb, Mechanical Tests+Effect of Temperature Alone on Deposition

Tensile test pieces have been brazed according to the same protocol as for example 2, but by making double metallisation of zirconia with titanium and then niobium. However, the titanium ring is not metallised. These test pieces are subjected to the tensile test and the rupture stress values are measured. For 6 test pieces, an average rupture stress of 42 MPa is obtained, which is significantly lower than the rupture stresses obtained with the test pieces in example 2.

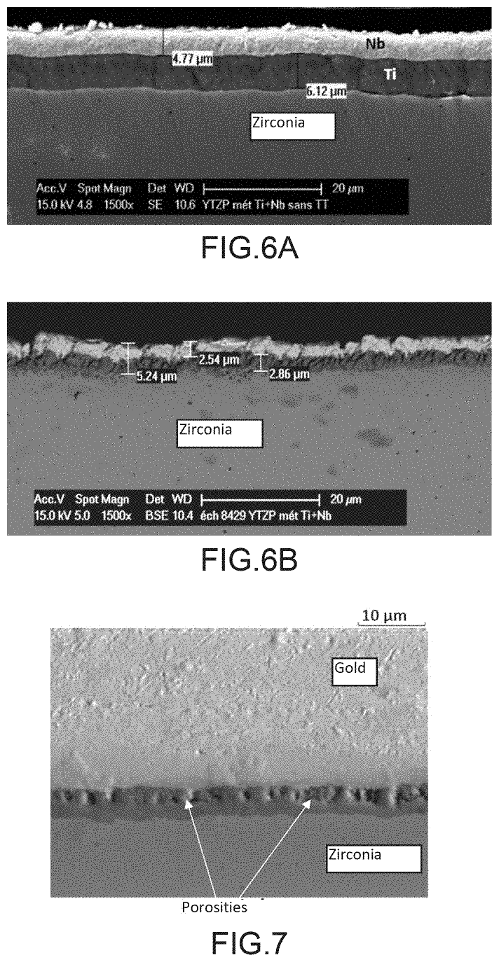

To understand this result, a heat treatment has been applied, identical to that of the brazing cycle, to the Ti+Nb metallised zirconia (without performing brazing). This treatment leads to a degradation of the deposit with the formation of porosities, which weakens the interface on the zirconia side. Scanning electron microscope pictures obtained before and after heat treatment are represented in FIGS. 6A and 6B respectively.

After brazing, these porosities are found at the zirconia side interface (FIG. 7), confirming that this interface represents the weak point of the assembly, namely it is the rupture place during mechanical tests.

The degradation of metallisation (presence of porosities) during the brazing cycle takes place even before the gold braze is melted. This leads to poorer interfaces than brazing without zirconia metallisation. Indeed, it seems that it is zirconia itself that releases oxygen which degrades the metallisation, the latter thereby becoming the weak point of the assembly.

This shows that the metallisation of zirconia does not result in an assembly with a good mechanical strength.

### REFERENCES

1 Agathopoulos, S. et al, ‘Interactions at Zirconia-Au—Ti Interfaces at High Temperatures’. Key Eng. Mater. 206-213, 487-490 (2001).

2 Agathopoulos, S. et al, ‘A review of recent investigations on zirconia joining for biomedical applications’. Advances in Joining of Ceramics (2003) 135-147.

3 Lei, Y. et al, ‘Evaluation of Biomedical Ti/ZrO₂ Joint Brazed with Pure Au Filler: Microstructure and Mechanical Properties’. Metals 10, 526 (2020).

4 Fischer M. et al, ‘Wetting and reactivity of zirconia by Au—Ti active alloys in view of zirconia/titanium brazing with pure gold’. Proceedings of the International Brazing and Soldering Conference 2021. 3-6 Oct. 2021.

5 FR3051131-A1 ‘Procédé de brasage d′un élément métallique sur une piéce de zircone et dispositif implantable brasé’

1. A method for assembling a zirconia part to a titanium element with braze, the method comprising the following steps:
coating a surface of the titanium element with a niobium layer,
positioning a braze between the zirconia part and the niobium layer, the braze being of gold or a gold alloy,
heating the whole to a temperature higher than the melting temperature of the braze and then cooling the whole, whereby an assembly comprising the zirconia part and the titanium element assembled by a brazing joint comprising a first portion of gold or a gold alloy, a second portion formed by a reaction layer comprising intermetallics of the AuNbTi system, and a third portion formed by an oxide reaction layer is obtained.

2. The method according to claim 1, wherein the braze is of gold.

3. The method according to claim 1, wherein the braze is a gold alloy comprising gold and titanium.

4. The method according to claim 1, wherein the braze is a gold alloy comprising gold, titanium and nickel.

5. The method according to claim 1, wherein the braze is a gold alloy comprising gold and zirconium.

6. The method according to claim 1, wherein the braze is a gold alloy comprising between 0.5 and 4% by mass of titanium or zirconium.

7. The method according to claim 6, wherein the braze is a braze having composition Au3Ni0.6Ti, AuxTi or AuxZr with x between 0.5 and 3% by mass.

8. The method according to claim 1, wherein the braze is a braze foil having a thickness of between 25 μm and 200 μm.

9. The method according to claim 1, wherein step d) is carried out under secondary vacuum, under reducing atmosphere or under neutral gas.

10. The method according to claim 1, wherein before coating the surface of the titanium element with a niobium layer and before positioning the braze, the method comprises a step of cleaning the zirconia part, the titanium element or the braze.

11. An assembly successively comprising:
a zirconia part,
a brazing joint comprising a first portion of gold or a gold alloy, a second portion formed by a reaction layer comprising intermetallics of the AuNbTi system, and a third portion formed by an oxide reaction layer,
a titanium element.

12. The assembly according to claim 11, wherein the brazing joint comprises a first portion of a gold alloy AuNiTi, AuTi or AuTiZr, a second portion formed by a reaction layer comprising intermetallics of the AuNbTi system, and a third portion formed by an oxide reaction layer.

13. The assembly according to claim 11, wherein zirconia is an yttria-stabilised zirconia.

14. The assembly according to claim 11, wherein zirconia is an alumina-zirconia composite.

15. A cochlear implant comprising an assembly as defined in claim 11, the brazing joint of the assembly comprising a first portion of gold or a gold alloy AuTi or AuTiZr, a second portion formed by a reaction layer comprising intermetallics of the AuNbTi system, and a third portion formed by an oxide reaction layer.
